(19) United States
(12) Patent Application Publication
(10) Pub. No.: US 2024/0115120 A1
(43) Pub. Date: Apr. 11, 2024

(54) DEVICES AND METHODS FOR CREATING UNIFORM ILLUMINATION

(71) Applicant: Intuitive Surgical Operations, Inc, Sunnyvale, CA (US)

(72) Inventor: Jonathan D. Halderman, Sunnyvale, CA (US)

(21) Appl. No.: 18/270,189

(22) PCT Filed: Jan. 6, 2022
(86) PCT No.: PCT/US2022/011424

§ 371 Date: Jun. 28, 2023

Related U.S. Application Data
(60) Provisional application No. 63/134,327, filed on Jan. 6, 2021.

(51) Int. Cl. A61B 1/07; A61B 1/04; A61B 1/06

(52) U.S. Cl. CPC A61B 1/07; A61B 1/042; A61B 1/0638; A61B 1/0655; A61B 1/0676

(57) ABSTRACT

With optical fiber-based illumination, uniform perceived illumination at an image capture device can be achieved with an input-angle-dependent intensity distribution at the input to the optical fiber. Disclosed are various apparatus and associated methods of operation that achieve such an angular intensity distribution, either statically or via programmable or controllable devices. Example embodiments utilize spatial intensity modulation of the light at a transform plane preceding the input to the optical fiber, light emission by multiple individually controllable emitters at different distances from or different angles relative to an optical axis, and/or beam sweeping across a range of input angles in synchronization with intensity control to generate the desired angular intensity distribution.

### CLAIM OF PRIORITY

This application claims the benefit of priority to U.S. Provisional Patent Application No. 63/134,327, filed on Jan. 6, 2021, which is incorporated by reference herein in its entirety.

### TECHNICAL FIELD

This disclosure relates to illuminating targets via optical fiber(s), and specifically to apparatuses and methods used to couple light into a proximal end of the optical fiber(s).

### BACKGROUND

During optical imaging of targets that are not easily accessible, such as interior organs and tissues, optical fiber(s) are often used to illuminate the target. In endoscopy, for example, an endoscope equipped with an optical fiber or fiber bundle and a camera at the distal end of the endoscope may be inserted through a natural body orifice or a small incision in the body. The endoscope may be guided, e.g., inside body lumens such as the gastrointestinal, respiratory, or urinary tract or through a surgical incision towards the anatomic site of interest (hereinafter referred to as a “target”). To illuminate the target, a distal end of the fiber(s) may be directed at the target, and light may be coupled into a proximal end of the fiber(s) and guided via total internal reflection to the distal end, from which the light may exit in all directions. The distal end of the optical fiber may thus function, in essence, as a point light source. Light reflected or backscattered off the illuminated target may be captured with the camera. It would be desirable for a diffusely reflecting or fluorescent target with uniform surface conditions that is perpendicular to the optical axis associated with the distal fiber end to appear with uniform brightness in the resulting image. However, the actually observed brightness, which corresponds to the intensity measured by the images sensor of the camera, may exhibit a maximum in the center region of the image and may fall off toward the outer image regions as a result of a decrease in the solid angle subtended by a target region of given size towards greater distance from the optical axis. This fall-off, which is not due to any differences in actual surface conditions across the target, can constitute an impediment to quantitative analysis of the fluorescence or absorbance behavior of the target, reducing the informational value of the image.

### SUMMARY

Described herein are apparatus, systems, and methods for fiber-based illumination that achieve uniform perceived illumination of a target by modifying the angular radiant intensity distribution at the input end of the illuminating fiber or fiber bundle (herein synonymous with the proximal fiber end). In various embodiments, to compensate for the fall-off in the measured intensity at an image sensor towards the outer image regions, the intensity of the light coupled into the fiber (bundle) at the proximal end is controlled as a function of input angle to create, at the output end (herein synonymous with the distal fiber end), a non-isotropic output radiant intensity distribution with an intensity that increases towards larger output angles.

Beneficially, creating uniform perceived illumination, which would result in a uniform observed intensity for a flat target of uniform optical surface properties, enables greater accuracy in identifying, locating, and monitoring features in the illuminated scene. Further, achieving uniformity in the perceived illumination by changing the angular radiant intensity distribution of the illuminating point light source can be preferable over compensating for non-uniformity by amplifying the sensor signal in a spatially dependent matter, as such non-uniform amplification can, for example, cause increased and spatially dependent noise levels. Aside from noise issues, sensor signal amplification may not even be available as a means to compensate for non-uniform illumination in some circumstances. For example, fluorescence imaging generally relies on a minimum excitation intensity to generate a fluorescent response detectable by the sensor. Thus, with the same amount of fluorescent marker (e.g., tagging malignant cells) present at the center of the field of view and at the edge of the field of view, non-uniform illumination may result in such low intensity at the edge of the field of view that the fluorescent marker at the edge of the field of view will not be detected, regardless of the amplification applied to the sensor signal. On the other hand, if the overall intensity of illumination is increased to compensate for the fall-off towards the edge, it is possible that the fluorescent marker “blooms” in the center of the field of view (meaning that it covers many more pixels than correspond to the actual marker location, making it hard to be clearly located) or is photobleached.

This disclosure describes multiple embodiments of apparatus that can provide light at the input to the optical fiber (bundle) as a function of input angle. In various embodiments, light from one or more light emitters (e.g., light emitting diode (LED) or other lasers) is collimated and then focused down onto the input end of the fiber (bundle). In these configurations, the intensity at the Fourier transform plane between the collimating and focusing optics can be varied as a function of distance from the optical axis to thereby vary the intensity in the focal region at the input end of the optical fiber as a function of the input angle. Alternatively, a beam sweeper preceding the collimating optic may be used to sweep light received from the light emitter(s) across the collimating optic, thereby changing the input angle as a function of time, and the intensity of the light may be controlled in synchronization with the sweep to change the intensity as a function of the input angle. In other embodiments, multiple light emitters are configured to emit light directly towards a common focal region at the input end of the optical fiber, but from different directions, and an input-angle-dependent intensity is achieved by controlling the relative optical output power of the different light sources, Multiple light emitters may also, alternatively, be used in conjunction with collimating and focusing optics, which facilitates despeckling in the Fourier transform plane.

The preceding summary is intended to provide a basic overview of the disclosed subject matter, but is not an extensive summary of all contemplated embodiments, nor is it intended to identify key or critical elements or delineate the scope of such embodiments.

### BRIEF DESCRIPTION OF THE DRAWINGS

The foregoing will be more readily understood from the following description of various example embodiments, in particular, when taken in conjunction with the accompanying drawings, in which:

FIG. 1 is a block diagram of an example image capture system that achieves illumination of a target via an optical fiber, in accordance with various embodiments.

FIG. 2A is a conceptual diagram of the locations of a light source and collocated image sensor relative to a target in an image-capture setup in accordance with various embodiments.

FIG. 2B shows an example desired uniform intensity distribution across the image sensor of FIG. 2A,

FIG. 2C shows an example actual intensity distribution across the image sensor of FIG. 2A for an isotropic light source.

FIG. 3 is a schematic explaining the radial fall-off in intensity observed in FIG. 2C.

FIGS. 4A-4E show example images of the light output of an optical fiber for various input angles of collimated light coupled into the fiber.

FIG. 5 shows graphs of the output intensity of the optical fiber as a function of output angle, as derived from the images of FIGS. 4A-4E.

FIG. 6 is a schematic diagram of an example illumination source for controlling the angular radiant intensity distribution of light coupled into an optical fiber by spatial filtering in a Fourier transform plane, in accordance with various embodiments.

FIG. 7A is a schematic diagram of an example illumination source including multiple light emitters at multiple angles relative to an optical fiber for controlling the angular radiant intensity distribution of light coupled into the optical fiber, in accordance with various embodiments.

FIG. 7B is a schematic diagram of an example illumination source including multiple light emitters at multiple angles for controlling the angular radiant intensity distribution of light coupled into the optical fiber in conjunction with optics for despeckling, in accordance with various embodiments.

FIG. 7C is a schematic diagram of an example illumination source including multiple light emitters emitting collimated light parallel to the optical axis of a focusing optic that focuses the light onto an optical fiber, in accordance with various embodiments.

FIGS. 7D-7F are schematic diagrams of example planar arrangements of light emitters of the illumination source of FIG. 7C, in accordance with various embodiments.

FIG. 7G is a schematic diagram of an example illumination source, otherwise as shown in FIG. 7C, which combines light at multiple wavelengths emitted by groups of light emitters placed at multiple respective planes, in accordance with various embodiments.

FIG. 8A is a schematic diagram of an example illumination source for controlling the angular radiant intensity distribution of light coupled into an optical fiber by scanning light from one or more light emitters across a collimating optic as the intensity of the light is varied, in accordance with various embodiments.

FIG. 8B is a schematic diagram of an example illumination source for controlling the angular radiant intensity distribution of light coupled into an optical fiber by scanning light from one or more light emitters partially across a collimating optic as the intensity of the light is varied, in accordance with various embodiments,

FIG. 9 is a flow chart of a method for illuminating a target, in accordance with various embodiments.

FIGS. 10A-10D are flow charts of various methods for controlling the angular radiant intensity distribution of light coupled into an optical fiber, in accordance with various embodiments.

### DESCRIPTION

Image-capture systems sometimes utilize optical fiber to guide light from an illumination source to an illumination target in scenarios where spatial or other constraints prevent the illumination source from being positioned to illuminate the target directly. An example application of such fiber-based illumination is endoscopy, which involves the insertion of an endoscope into the body to access an anatomical target such as an internal organ or tissue for visual observation endoscope may include a housing for the illuminating fiber as well as a camera at the distal fiber end to capture light reflected off the target. Similar optical devices, generally referred to as borescopes, may be used in various industrial contexts for quality testing and inspection of (non-anatomical) targets in otherwise inaccessible areas, inside pipes, engines or other machines, etc.

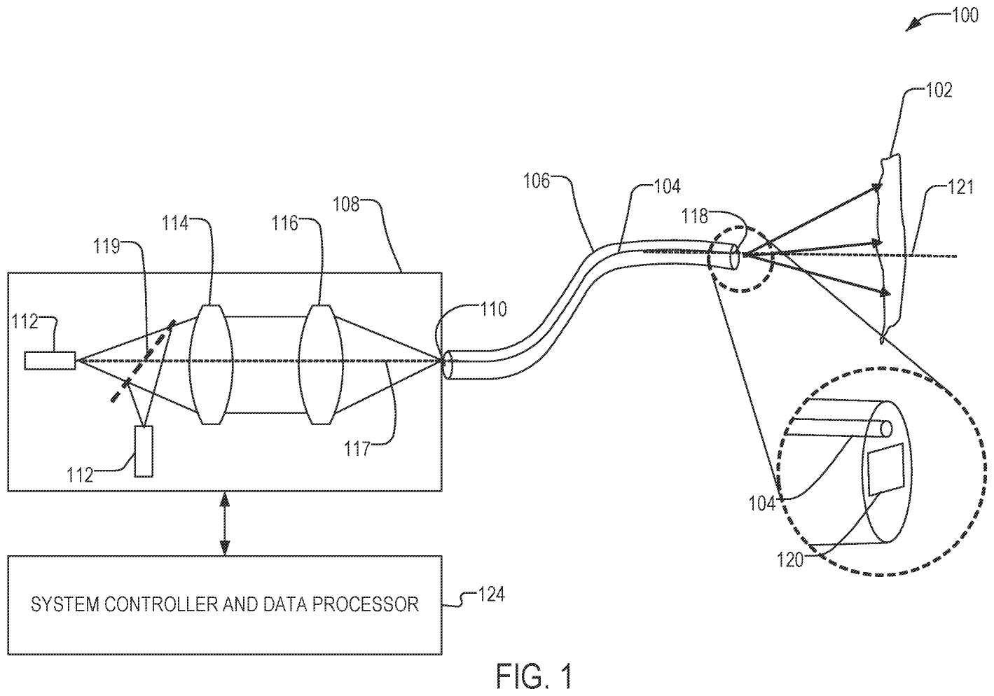

FIG. 1 is a block diagram of an example image capture system 100 that achieves illumination of a target 102 via an optical fiber or fiber bundle 104, in accordance with various embodiments. The fiber or fiber bundle 104 is housed in rigid or flexible tube 106, such as, e.g., a catheter, which may extend from an output of an illumination source 108 to a location near the target 102. For ease of reference, the following description refers simply to an optical fiber 104. However, it is to be understood that an optical fiber bundle (e.g., forming, along with protective sheathing, a fiber-optic cable) may often be used The description is, in general, equally applicable to both single-fiber and fiber-bundle embodiments. It is assumed, for example, that the individual fibers within a fiber bundle share a common proximal end and a common distal end.

The illumination source 108, which is configured to generate and couple light into the optical fiber at the proximal fiber end (or input end) 110, may include one or more light emitters 112, such as lasers (e.g., diode lasers), light emitting diodes (LEDs), or broadband light sources, and (optionally) optics to direct the light into the optical fiber 104. As shown, the optics may, for instance, include a collimating optic 114 that turns a diverging beam of light received from the light emitters 112 into a collimated beam of parallel light, as well as a focusing optic 116 that focuses the light down onto a region at or very near the input end 110 of the fiber 104. The fiber input end 110 may, in other words, be placed substantially at the focal plane of the focusing optic 116. The collimating and focusing optics 114, 116 may, as shown, share a common optical axis 117 with the input end 110 of the fiber as well as with a diverging beam of light received by the collimating optic 114 and the focused beam of light entering the optical fiber. The collimating and focusing optics 114, 116 may generally be or include refractive and/or reflective optical components, such as lenses and/or (spherical or parabolic) mirrors. To facilitate illumination at different wavelengths, the collimating optic 114 may receive and combine light from multiple light emitters 112 emitting at different wavelengths, with one or more beamsplitters 119 in the optical path serving to direct the light from light-generating devices 112 towards the collimating optic 114. Light coupled, by the illumination source 108, into the fiber 104 at the proximal end 110 leaves the fiber 104 at the distal end (or output end) 118, forming a diverging beam that, in use, is directed at the target 102. The distal fiber end 118 may thus function as a point light source for illumination of the target 102.

The image capture system 100 includes one or more cameras to acquire images of the illuminated target 102. Each camera includes suitable imaging optics (e.g., a lens, not shown) and an image sensor 120 (shown in the detail view), e.g., implemented as a photodiode array that detects light reflected or diffusely scattered off the target 102 or fluorescently emitted by the target 102. Multiple cameras, such as two cameras, may be used to provide for a stereoscopic view of the target 102. The camera and distal end 118 of the illuminating optical fiber 104 may be configured, in their relative position and orientation, for substantially coaxial illumination, meaning that the optical axis of the camera (more precisely, the optical axis of the imaging optic of the camera) and the optical axis of illumination (which, for a point source created at the distal end 118 of a single optical fiber 104, generally corresponds to the optical axis 121 of the fiber 104 at the distal end 118) coincide or at least nearly coincide. Coaxial illumination can generally be achieved if the camera is collocated with the distal fiber end 118 and both are oriented towards the same region of the target. While coaxial illumination has advantages for illumination uniformity, it is also possible in principle to use oblique illumination, where the optical axis of the camera and the optical axis of illumination enclose a substantial angle. In either coaxial or oblique illumination, the field of view of the camera and the field angle of illumination may be matched for maximum efficiency.

In various embodiments, as shown, a camera (as symbolically represented by the image sensor 120) is placed in the tube 106, adjacent the distal fiber end 118. To provide room for the camera, the tube 106 may, at the distal end, expand into a larger-diameter housing. In some embodiments, to provide for substantially coaxial illumination, the camera is substantially collocated with the distal end 118 of the optical fiber 104. For example, the imaging optic of the camera and an output face of the optical fiber 104 at the distal fiber end 118 may lie side by side in a plane perpendicular to the optical axis 121 of the fiber 104 at the distal end 118, at a distance from each other that is significantly (e.g., one or more orders of magnitude) smaller than the distance between the distal fiber end 118 and the target 102 (such that, from the perspective of the target 102, the distal fiber end 118, which may act as a point light source for illumination, and the image sensor 120 are practically at the same location). As another example, the individual optical fibers of a fiber bundle 104 may, at the distal end, split up, e.g., to form a ring concentrically surrounding the camera, or two or more noncontiguous light-emitting areas distributed around the camera (e.g., two half-moon-shaped portions on opposite sides of the camera), with the diameter of the ring or the distance between the light-emitting areas being, again, small compared to the distance between the fiber end 118 and the target 102 such that the individual fibers collectively still form a point light source. For purposes of the present disclosure, the distal end(s) 118 of the optical fiber(s) 104 and the camera, placed at substantially the same distance from the target, are deemed “substantially collocated” if the largest angle between the optical axis of the camera (which determines where the center of the field of view is on the target) and the direction of illumination, understood to be the direction defined between the light source formed at the distal fiber end 118 of any of the optical fibers (104) and the center of the field of view on the target, is no more than 15°. As will be appreciated, staying below this angular threshold ensures that the optical axes of the camera and the optical axes of the optical fiber ends (whether enclosing a slight angle or being parallel but slightly displaced) at least nearly coincide, thus achieving “substantially coaxial illumination”.

Returning to the description of the image capture system at large, the image sensor 120 of the camera is communicatively coupled, e.g., via electrical wires running alongside the optical fiber 104 inside the tube 106, to suitable electronic circuitry for reading out information from the image sensor 120 and processing the image data. In Some embodiments, such image-processing functionality is combined with functionality for controlling the illumination source in a system controller and data processor 124, which is generally implemented with a suitable combination of computational hardware and/or software. For example, the system controller and data processor 124 may be a general-purpose computer running suitable control and image-processing software, or may utilize, alternatively or additionally, a suitably configured special-purpose processor (such as, e.g., a graphical-processing unit (GPU), field-programmable gate array (FPG), application-specific integrated circuit (ASIC), or digital signal processor (DSP)). General-purpose computers including one or more central processing units (CPUs) are, for instance, sometimes augmented with hardware accelerators (e.g., using a GPU) customized to perform complex, but fixed image-processing tasks. In addition to one or more general and/or special-purpose processors, the system controller and data processor 124 may have one or more machine-readable storage devices, which may include both volatile memory (such as random-access memory (RAM)) and non-volatile memory (such as read-only memory (ROM), flash memory, or magnetic or optical computer storage devices), Further, the system controller and data processor 124 may include input/output devices (e.g., keyboard, display) and network interfaces for communicating with human operators as well as other computers.

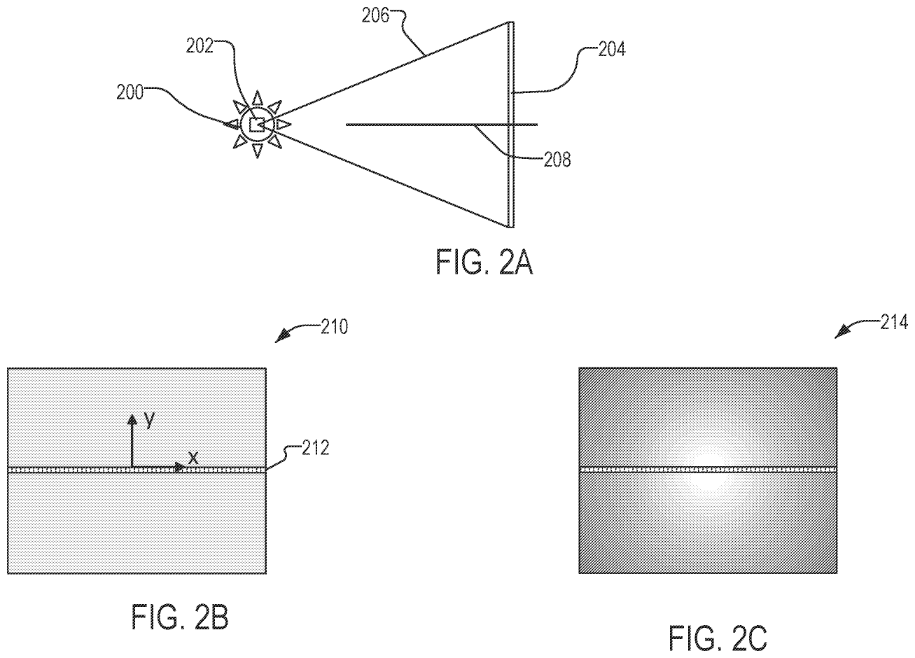

With reference to FIGS. 2A-5, the perceived illumination of a flat target will now be described to illustrate both the non-uniform illumination obtained with conventional illumination sources and the underlying principles for achieving a desired uniform illumination in accordance with the disclosed subject matter.

FIG. 2A is a conceptual diagram of the locations of a light source 200 and collocated camera, symbolically represented as an image sensor 202, relative to a target 204 in an image-capture setup in accordance with various embodiments. The light source 200 is assumed to be a point light source, characterized by uniform radial intensity in all directions, although, in practice, the light source generally has an associated aperture that limits emission, e.g., to a semispherical or substantially conical region. In any case, the light source 200 is positioned to emit light in a general direction towards the target 204, FIG. 2A indicates the portion of the light 206 emitted from the light source 200 that illuminates the target 204. (Note that the depictions of the light source 200 and image sensor 202 are symbolic and, as such, fail to reflect that both light source 200 and image sensor 202 face the target 204.) In many application scenarios, the target 204 may be assumed to be a flat Lambertian screen, meaning that it has an ideal diffusely reflecting surface that, when illuminated uniformly, appears equally bright from any angle. The radiant intensity of a Lambertian screen (i.e., the optical power it emits per unit solid angle, measured in Watts per steradian) is directly proportional to the cosine of the angle between the direction into which the light is reflected and the surface normal 208 to the screen. Alternatively, in some scenarios, the target 204 can be assumed to be an isotropic screen, which, when illuminated uniformly, emits light with the same radiant intensity in all directions. Such uniform emission can result, e.g., from a uniform distribution of a fluorescent material across the screen.

FIG. 2B shows an example of a desired uniform intensity distribution 210 across the image sensor 202 of FIG. 2A. The image sensor 202 may include a regular array (e.g., rectangular in shape) of uniformly sized sensor elements, whose acquired signals form the pixels of the image; for simplicity, the sensor elements themselves are hereinafter referred to as “pixels.” For purposes of the following discussion, the imaged region within the target 204 is conceptually broken down into a rectangular grid of sub-regions, herein “cells,” that equal the pixels in number. Imaging optics (not shown) associated with the image sensor 202 effectively maps each cell within the imaged region onto a corresponding pixel within the image. FIG. 2B indicates a single row 212 of pixels along the horizontal dimension (herein denoted as the x coordinate), vertically at the center of the image. The image sensor 202 includes multiple such pixel rows along the vertical dimension (denoted the y-coordinate). If the optical surface properties of the target 102 are uniform, i.e., the same for all cells (as is the case for, e.g, a Lambertian screen or a uniformly fluorescent or other isotropic screen), it would be desirable for the captured image to reflect this uniformity with equal signal amplitudes (e.g., gray-scale values) across all pixels, as shown by the intensity distribution 210,

FIG. 2C shows an example of an actual intensity distribution 214 across the image sensor 202 of FIG. 2A for an isotropic light source 200, that is, a light source 200 that emits with uniform radiant intensity in all directions. As can be seen, the intensity is maximum at the center of the image, and falls off radially.

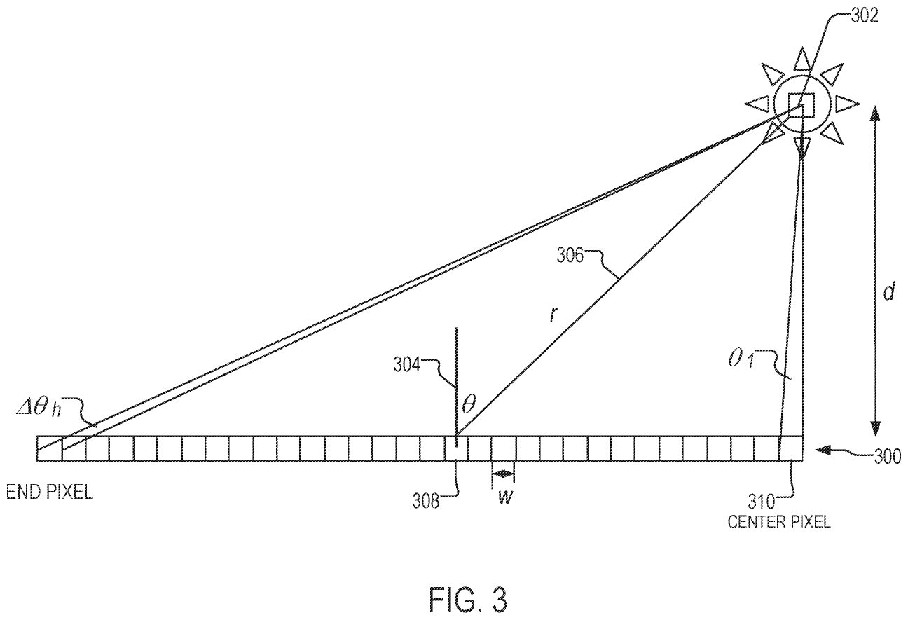

FIG. 3 is a schematic explaining the radial fall-off in intensity observed in FIG. 2C. Consider cells along a horizontal row 300 within the target, corresponding to pixels along a center row 212 within the image acquired by an image sensor 202 located at point 302. Let d be the normal distance between target and sensor. (It is assumed, for purposes of this discussion, that the spatial extent of the aperture stop of the imaging optic associated with the image sensor 220 is negligible as compared to the normal target-sensor distance d and the width of the target 102.) Further, let θ be the angle between the normal 304 to the target and a line of sight 306 between the sensor at 302 and a given cell 308. The distance r between the cell 308 and the sensor at 302 equals d/cos θ. For a fixed cell height h, the vertical angle subtended by cell 308 is

$$Δ {θ}_{v} \approx \frac{h}{r} = \frac{h cos θ}{d} ;$$

that is, the subtended vertical angle (or “angular height”) of the cells drops from h/d at the center (cell 310) as cos θ. For a fixed cell width w, the horizontal angle subtended by cell 308 is

$${Δθ}_{h} \approx \frac{w cos θ}{r} = \frac{{w ( cos θ )}^{2}}{d} ;$$

that is, the subtended horizontal angle (or “angular width”) of the cells along drops from h/d at the center (cell 310) as cos θ)². The extra factor of cos θ results from the projection of the cell onto a direction perpendicular to the line of sight 306. Combining the drop-off in angular height and width, it can be seen that the solid angle subtended by the cells drops along the horizontal direction as (cos θ)³ from a cell 310 at the center of the imaged region. The same analysis applies to a vertical strip of cells corresponding to a center column with the acquired image. Accordingly, the solid angle subtended by the cells within the imaged region, which are mapped onto pixels of the image, falls as (cos θ)³ with radial distance from the image center.

An ideal point light source at point 302 (e.g., as approximately provided by the distal end of an optical fiber 104) emits light uniformly in all directions, that is, with an isotropic radiant intensity (measured in Watts/steradian). The amount of light received by any cell 308 of the target is, therefore, proportional to the solid angle subtended by the cell 308, and thus proportional to (cos θ)³. Conversely, considering now the illuminated cell 308 as a point light source of reflected light (as it would be for an isotropic screen), the fraction of light emitted by the cell 308 that is received at the corresponding pixel of the image sensor 220 is proportional to the solid angle subtended by the pixel (from the perspective of the cell 308) which is proportional to (cos θ′)³, where θ′ is the angle between a normal to the sensor and the line of sight 306 between the sensor and the cell 308. Assuming that the sensor normal is parallel to the normal to the target, θ′=θ. Thus, the light received at the image sensor from, e.g., a uniformly fluorescent target illuminated by an isotropic light source varies with (cos θ)⁶, resulting in the observed radial fall-off in image intensity shown in FIG. 2C. For an imaged region that spans, from the center to the outer edge, an angle of, for example, 30°, a cell at the edge would receive only about 65% of the optical power that a cell at the center would receive, and the corresponding pixel at the edge of the image sensor would receive only 65% of the fraction of the reflected light that the center pixel would receive from the center cell; as a result, the intensity measured by a pixel at the edge would be only about 42% of center intensity. For a Lambertian screen, light emitted from a pixel to the sensor at angle θ′=θ would collect another factor of cos θ, for a radial fall-off in image intensity of (cos θ)⁷.

In accordance with various embodiments, the above-described angular dependence of the fraction of light that reaches a certain region of the target and is then returned from that target region to the image sensor is compensated for by creating a non-isotropic light source with an angular radiant intensity distribution, More specifically, the radiant intensity of the light source at the end of the optical fiber is modified to increase towards larger output angles, in a manner such that the increased emitted intensity at larger output angles makes up for the smaller fraction of light that illuminates corresponding target regions at larger radial distance from the center and is reflected back or fluorescently emitted towards the sensor by those corresponding target regions. Such an angular radiant intensity distribution can be achieved by exploiting the dependence of the angular distribution of light output at the distal end of the optical fiber on the input angle of light launched into the optical fiber at the proximal end.

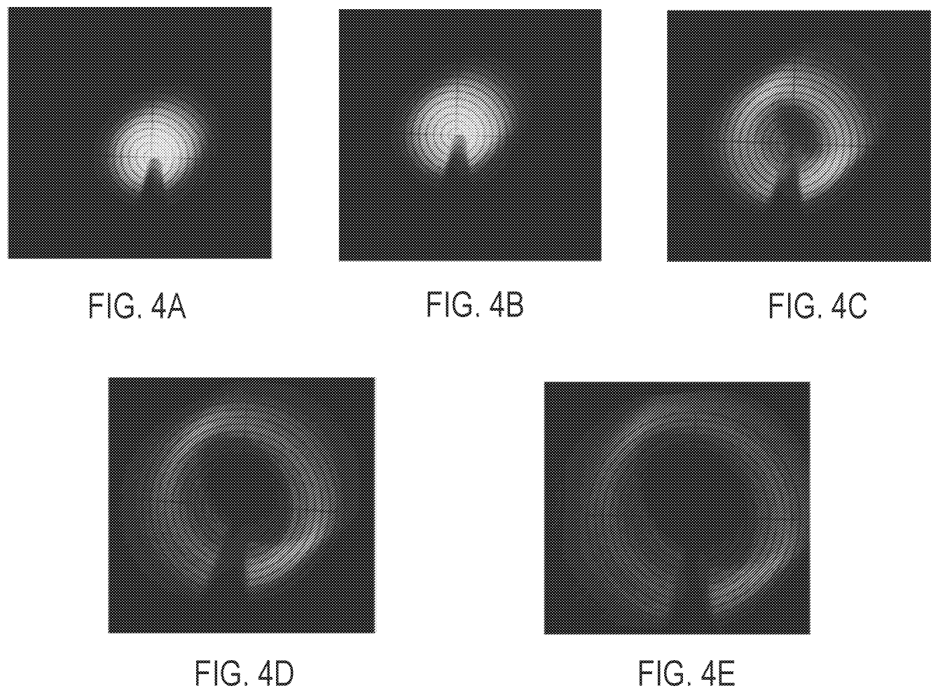

FIGS. 4A-4E show example images of the light output of an optical fiber for various input angles of collimated light coupled into the fiber. As can be seen in FIG. 4A, light input at 0°, that is, along the optical axis of the fiber at the input end, results in a bright central spot at the output, corresponding to an angular radiant intensity distribution that is maximum at 0°, that is, in a direction along the optical axis of the fiber at the output end. For a small input angle of 10°, as shown in FIG. 4B, the output light still forms a central spot. For 20°, 30°, and 35° input angles, however, the images in FIGS. 4C-4E show a bright ring with increasing diameter towards increasing input angle, reflecting a shift in the maximum of the angular radial intensity distribution at the output to increasing output angles. To understand the annular (i.e., ring-shaped) intensity output of the optical fiber, consider rays of light incident upon the fiber. Meridional rays, which enter in a plane that includes the optical axis of the optical fiber, will travel along the fiber within that plane, guided by total internal reflection. On the other hand, skew rays, which enter the fiber within a plane that does not include the optical axis, will hit the core-cladding interface of the fiber at oblique angles and propagate along a generally helical path. For a bent fiber, the path lengths of these helical rays are generally different, which randomizes their output angle, creating a cone of output light.

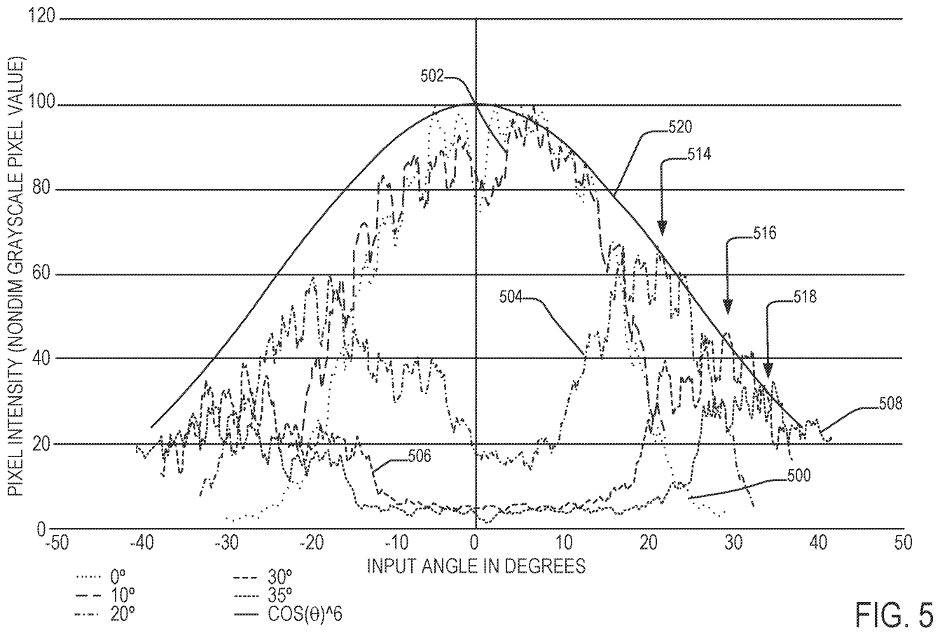

FIG. 5 shows graphs 500, 502, 504, 506, 508 of the output intensity of the optical fiber as a function of output angle 9, as derived from the images of FIGS. 4A-4E for the various input angles. The output intensity, here given as pixel grayscale values, is plotted along a row of pixels through the center of the respective images (corresponding to a diameter through the radially symmetric intensity distribution), with positive and negative output angles corresponding to opposite directions away from the center. For input angles of 20°, 30°, and 35°, the graphs 504, 506, 508 show a double-peaked distribution with peaks 514, 516, 518 at output angles generally matching the input angles at which light is launched into the fiber. For example, the graph 504 for an input angle of 20° exhibits maxima at output angles of approximately ±20°. The angular full width at half maximum of the peaks is about 30° for the single central peaks at 0° and 10° input angles, and about 15° for each peak of the double-peaked distributions at the three larger input angles. FIG. 5 also includes a curve 520 representing an intensity fall-off (cos θ)⁶, which, as can be seen, forms an approximate envelope over the graphs 500, 502, 504, 506, 508.

As FIG. 5 illustrates, the angular radiant intensity distribution at the output end of an optical fiber can be controlled via the angular intensity distribution of light launched into the fiber at the input end. With coaxial illumination, light coupled into the optical fiber at any given angle generally results in an approximately circular illuminated region, with a diameter of the circle that increases with input angle, and multiple such circular illuminated regions having different diameters corresponding to different input angles can be combined to create an overall uniform illumination. With oblique illumination, the circular symmetry is broken, with light coupled into the optical fiber at any given angle resulting, e.g., in an approximately elliptical illuminated region. At least in principle, the illuminated (e.g., elliptical) regions for multiple input angles can be combined to improve the uniformity of illumination, although the loss of symmetry complicates determining the input angular intensity distribution that will result in a desired output angular intensity distribution. In the following, various embodiments of an illumination source for achieving a controllable angular intensity distribution at the fiber input are described.

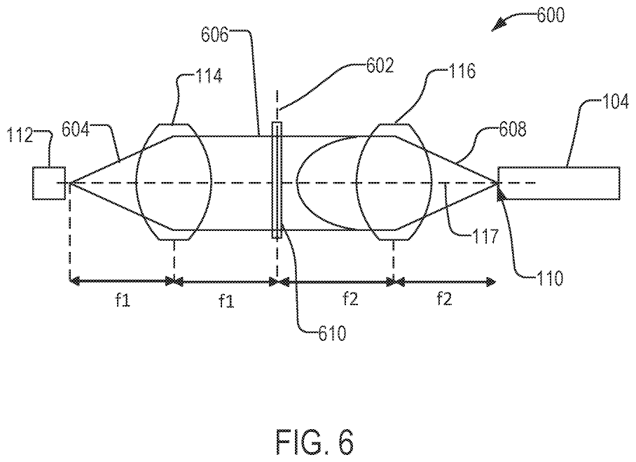

FIG. 6 is a schematic diagram of an example illumination source 600 for controlling the angular intensity distribution alight coupled into an optical fiber 104 by spatial filtering in a transform plane 602 (e.g., a Fourier transform plane), in accordance with various embodiments. The illumination source 600 includes at least one light emitter 112, such as, e.g., an LED or other laser, a collimating optic 114 (e.g., a lens), and a focusing optic 116 a lens). The collimating optic 114 may be placed at a distance from the light emitter 112 approximately equal to its focal length ƒ₁ so as to convert diverging light 604 received from the light emitter 112 into collimated, i.e., parallel light 606. The focusing optic 116 may be placed at a distance from the input end 110 of the optical fiber 104 approximately equal to its focal length ƒ₂ so as to re-focus the parallel light at the fiber input 110.

Lenses and other focusing optics effect a physical (as opposed to computational) Fourier transform of incoming light between the spatial and spatial-frequency (or wavevector) domains in that they map parallel light incident upon the optic from different directions (modeled as plane waves with different wavevectors) onto different respective spatial locations in the back focal plane and, conversely, map light coming in from different spatial locations in the front focal plane onto parallel outgoing light propagating in different directions. For example, a plane wave propagating parallel to the optical axis maps onto a focal point on the optical axis, and plane waves propagating at an angle relative to the optical axis map onto focal points in the focal plane at a radial distance from the optical axis that increase with angle. To exploit this property of focusing optics to control the angular distribution of the light 608 incident upon the input end 110 of the optical fiber 104 the illumination source 600 is configured such that the back focal plane of the collimating optic 114 (which is a plane parallel to the plane of the collimating optic 114 placed at a focal length ƒ₁ following the collimating optic 114) coincides with the front focal plane of the focusing optic 116 (which is a plane parallel to the plane of the focusing optic 116 placed at the focal length ƒ₂ preceding the focusing optic 116) in plane 602. The spatial intensity distribution in that plane 602 is the Fourier transform of the spatial-frequency distribution at the front focal plane of the collimating optic 114 as well as of the back focal plane of the focusing optic 116; the plane 602 is therefore also referred to as the “Fourier transform plane” or simply “transform plane,” By controlling the spatial intensity distribution of the light 606 in the transform plane 602, the angular distribution of light 608 incident upon the optical fiber 104 can be controlled.

To control or adjust the spatial intensity distribution of the incoming collimated light 606, the illumination source 600 includes a light modulator 610 positioned at the transform plane 602 between the collimating and focusing optics 114, 116, The light modulator 610 may be configured to create a radially varying intensity distribution that increases from the center at the optical axis 117 towards greater radii. This intensity variation along the radial direction may be continuous or, alternatively, discrete, taking the form of multiple concentric rings that increase step-wise in intensity. Various devices that may serve as the light modulator 610 are known to those of ordinary skill in the art.

In some embodiments, the light modulator 610 may be a diffractive optical element (also sometimes “diffractive element”), which may be a thin phase element designed to produce an arbitrary desired spatial intensity distribution by means of interference and diffraction, using either binary or analog, continuous phase profiles. Diffractive elements are commonly used as beam shapers or diffusers/homogenizers, and can be made from various materials and using various methods, including, e.g., depositing polymer on a glass substrate, etching hard semiconductor materials or fused silica, or embossing or injection-molding plastic. An example of commercially available diffractive elements are the Engineered Diffusers™ by RPC Photonics, Inc. (Rochester, NY). A diffractive optical element designed to create an inverse Gaussian beam profile from incoming light may be suitable as the light modulator 610, as the inverse Gaussian profile is a good approximation of a profile increasing at 1/(cos θ)⁶ in intensity towards greater radii. Alternatively, a diffractive optical element custom-made to achieve the 1/(cos θ)⁶ or 1/(cos θ)⁷ profile, as needed, may be used.

Another option for the light modulator 610 is a gradient-absorbing filter, designed to operate at the wavelengths of the light emitter(s), that provides spatially variable light transmission and absorption. The transmission profile of such a filter results from a varying optical density achieved, e.g., by dielectric and/or metallic coatings applied to a glass substrate. Suitable gradient-absorbing filters are commercially available, for instance, from Reynard Corporation (San Clemente, CA). For example, Bullseye® Apodizing filters from Reynard Corporation provide a Gaussian optical density distribution that decreases from the center to the edge, transmitting more light at greater radii. Alternatively to using a filter with a Gaussian distribution, gradient-absorbing filters, like diffractive elements, can also be made with a custom spatial profile.

Yet another option for the light modulator 610 is a prof; pan spatial filter made from a material that is controllably transmissive in a desired wavelength range (e.g., the visible and/or ultraviolet regime). A programmable spatial filter may, for example, include a liquid crystal material disposed between two optically transmissive plates, and electrically conductive and optically (or UV) transmissive layers (e.g., of indium tin oxide) disposed on the plates that are structured to form electrodes creating multiple individually addressable regions (or pixels) within the liquid crystal layer. The transmissivity of the liquid crystal in these regions can be adjusted via application of an electrical voltage across the liquid crystal layer in each region. The programmable spatial filter, thus, includes multiple variably transmissive and individually controllable elements, along with electronic circuitry for addressing these elements. In some embodiments, these elements form annular regions about the optical axis 117, each associated with a different range of illumination angles.

Beneficially, using a programmable spatial filter as the light modulator 610 allows calibrating the light modulator 610, or adjusting its operation in use, to optimize the resulting angular intensity distribution of the illuminating light. For calibration, the angular radiant intensity distribution of the light may, for example, be measured at the output of the illumination source 600 (corresponding to the input to the optical fiber 104) or at the output of the optical fiber 104, and serve as feedback to adjust the voltages applied at the spatial filter to create a desired (e.g., 1/(cos θ)⁶ or 1/(cos θ)⁷) angular intensity distribution. Alternatively, the intensity distribution of light reflected by a target, as measured by a camera that is, e.g., collocated with the output end 118 of the optical fiber 104, may be used directly as feedback for achieving uniform perceived illumination. Control of the programmable spatial filter based on the measurement may be performed by a suitable algorithm, e.g., similar to autoexposure algorithms as often used in digital photography, implemented on system controller and data processor 124 or another suitable computing device. In some embodiments, adjustments of the radiant intensity distribution at the fiber output via the light modulator 610 are combined with three-dimensional (3D) topology measurements of the target 102. In a simple example, the illuminating light source and the image sensor are placed at the center of an integrating sphere. The solid angle of all cells along a row of the sphere will be identical cells will also be perpendicular to the line of sight between the light source and the wall of the integrating sphere. So a point light source will create even illumination on every cell in the integrating sphere. When a cell reflects light back onto the aperture stop of the imaging system, the solid angle from the cell to the aperture stop is constant for all reflecting cells. However, the aperture stop will be foreshortened, as seen by the reflecting cell, by the cosine of the angle θ between cell line of sight to aperture stop and imaging system optical axis. So, in this case, the optimum illumination distribution is 1/cos(θ) to achieve even perception of illumination on the inside of the sphere.

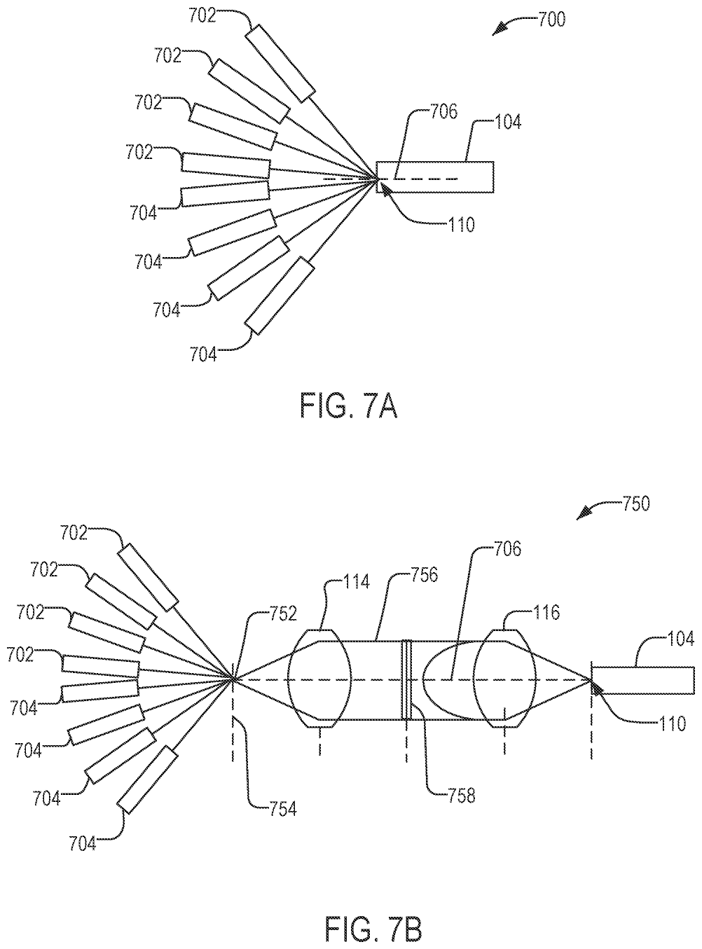

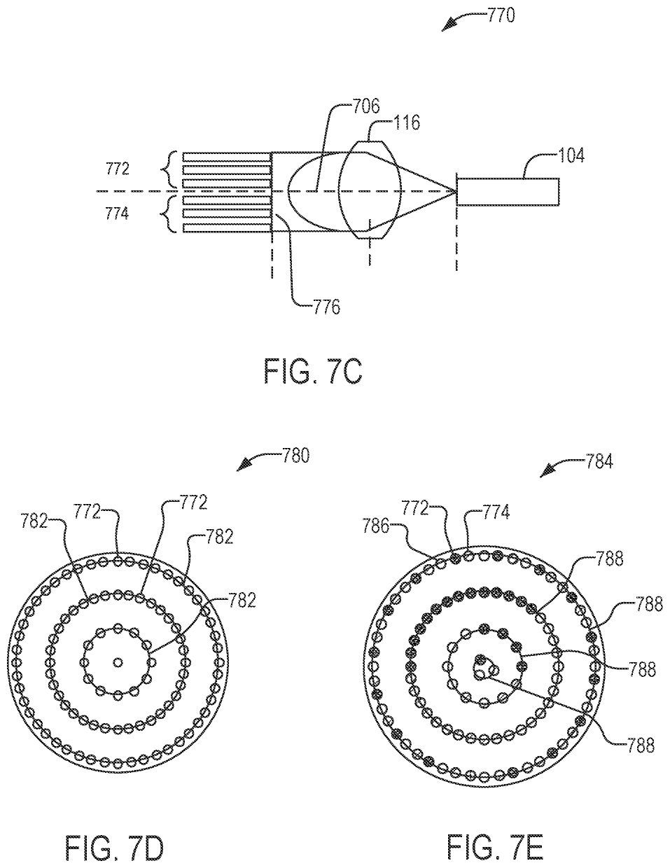

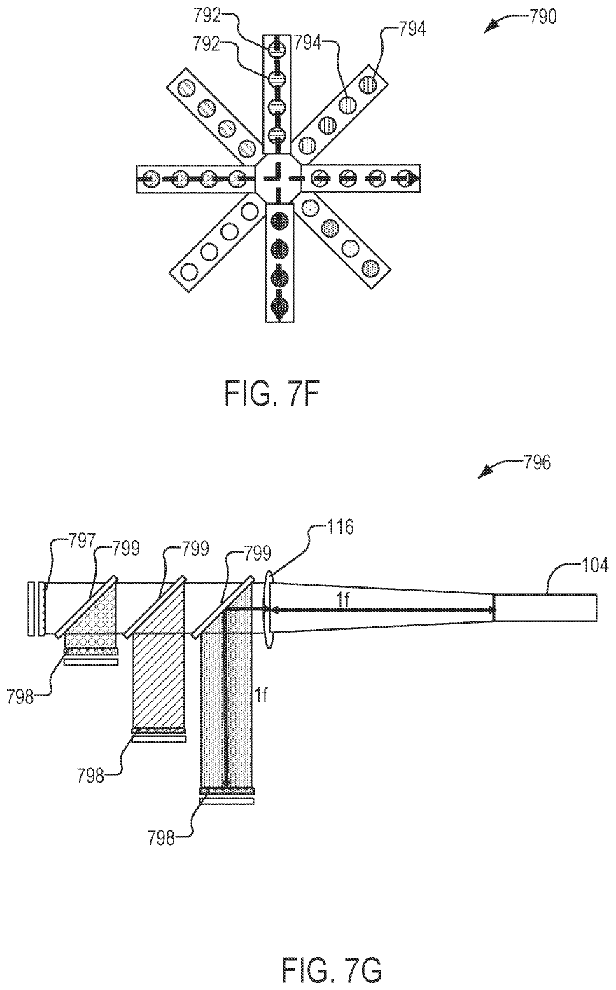

FIG. 7A is a schematic diagram of an example illumination source 700 including multiple light emitters 702, 704 at multiple angles relative to an optical fiber 104 for controlling the angular intensity distribution of light coupled into the optical fiber 104, in accordance with various embodiments. In this implementation, the illumination source 700 might not include collimating and focusing optics to direct the light at the fiber input. Instead, the light emitters 702, 704 (e.g., lasers or LEDs) may be oriented to emit light directly at the input end 110 of the optical fiber 104, at different angles relative to the optical axis 706 of the illumination source 700, which in use coincides with the optical axis of the fiber 104 at the input end 110. The light emitters 702, 704 may include groups of emitters that generate light at multiple respective wavelengths, allowing those wavelengths to be combined at the fiber input with minimal or no optics. For example, one group of light emitters 702 may emit green light while another group of light emitters 704 emits blue light. Within each group, the light emitters 702 or 704 can be independently varied in output intensity. Such intensity variation can be achieved, for instance, by directly changing the optical output power of each emitter (e.g., by adjusting the electrical power supplied to a diode laser, or adjusting the gain of a pumped laser via the power to the pump laser), or by using a controllable amplitude modulator at the laser output to variably reduce the intensity. Independently varying the output intensity of each emitter allows controlling (separately at each wavelength) the relative intensities of light at various input angles, and thus the angular intensity distribution at the fiber input. In various embodiments, at least three, or at least five, light emitters are used at each wavelength to achieve sufficient angular resolution in the controllability of the angular intensity distribution. Beneficially, light launched into the fiber at a discrete angle tends to be “blurred” around that angle, such that a discrete angular intensity distribution at the input causes a desired continuous angular intensity distribution at the output.

As shown, each group of light emitters 702 or 704 may be arranged in a plane containing the optical axis 706. Collectively, the light emitters 702 or 704 of the group cover a range of input angles, e.g., between zero or close to zero and a maximum input angle that may be determined based on the beam divergence desired at the output end 118 of the optical fiber 104 to fully illuminate a target region of interest. Since light coupled into the optical fiber 104 at a given angle from a single direction tends to be distributed in the optical fiber 104 between the input and output ends 110, 118 to result in an annular, conical light output, a group of light emitters 702 or 704 can be confined to one side of the optical axis 706 while still achieving a substantially cylindrically symmetric output. The other side of the optical axis 706 may be used for a group of light emitters of a different wavelength. For instance, in the depicted example, the green light emitters 702 may be placed above the optical axis 706 with the blue light emitters 704 being placed below the optical axis 706. Moving out of the plane of the figure, it is possible to add light emitters at other wavelengths, e.g, red, ultraviolet, and/or near infrared light.

As will be appreciated by those of ordinary skill in the art, the depicted arrangement of each group of light emitters (e.g., 702, 704) in a single quarter plane is only one of various possible configurations. Alternatively, light emitters for different wavelengths may be interspersed, e.g., in an alternating or (for three or more wavelengths) cyclical fashion, along angles in a half plane. Light emitters of the same wavelength at opposite sides of the optical axis 706 may be slightly shifted in their angular orientation to increase the number of input angles. For example, six blue light emitters may be placed at −40°, −20°, 0°, 10°, 30°, and 50° with five green emitters placed at −50°, −30°, −10°, 20°, and 40°. Further, the light emitters of a given wavelength need not necessarily be arranged in a single plane. Alternative configurations include, for example, an arrangement forming a “conical spiral,” where the azimuthal position of the emitter about the optical axis 706 changes along with the angle enclosed between the emitter axis and the optical axis 706.

An illumination source 700 including individually intensity-controllable emitters 702, 704 at different angles can be calibrated and operated similarly to an illumination source 600 with a light modulator including a programmable spatial filter. For example, the relative intensities of the different emitters can be determined based on measurements of the resulting angular radiant intensity distribution of light at the fiber input or the fiber output, or based on direct observation of the illumination of the target, optionally in conjunction with SD topology measurements of the target. A feedback control loop can be implemented using autoexposure or similar algorithms executed on a computing device such as, e.g., the system controller and data processor 124.

FIG. 7B is a schematic diagram of an example illumination source 750 including multiple light emitters 702, 704 at multiple angles in conjunction with collimating and focusing optics 114, 116 allowing despeckling, in accordance with various embodiments The light emitters 702, 704 direct their outputs onto a focal region 752 in the front focal plane 754 of the collimating optic 114. The collimated light 756 is refocused, by the focusing optic 116, onto the input of the optical fiber 104, at input angles that equal the respective angle of light emission relative to the optical axis 706 for each light emitter 702, 704 if the focal lengths of the collimating and focusing optics 114, 116 are equal. If the focal lengths differ, the tangent of each input angle equals the tangent of the respective emission angle by the light emitter 702 or 704, multiplied by the ratio of the focal lengths of the collimating and focusing optics 114, 116. Thus, by controlling the intensity of the emitters 702, 704 as a function of their respective angles relative to the optical axis 706, the angular intensity distribution at the input of the optical fiber 104 can be directly controlled, similar to the illumination source 700 shown in FIG. 7A. Local interference between light from multiple emitters 702 or 704 sharing a common emitted wavelength can cause a laser speckle pattern. Such laser speckle can be diminished with a laser speckle reducer 758 placed at the transform plane between the collimating and focusing optics 114, 116, A speckle reducer 758 can be implemented by an oscillating or other moving diffuser, for example, a light-weight diffuser carried on a thin elastic membrane that is actuated to undergo small circular motion in the transform plane, or a glass diffuser mounted in a steel structure brought into resonance via a pulsed magnetic field. Speckle reducers are generally known to those of ordinary skill in the art as well as readily commercially available. Similar to the illumination source 700 shown in FIG. 7A, the depicted arrangement of each group of light emitters (e.g., 702, 704) in a single quarter plane for the illumination source 750 shown in FIG. 7B is only one of various possible configurations, and the illumination source 750 may use other arrangements of light emitters described above. Moreover, the illumination source 750 may include more than two groups of light emitters, such as green, blue, and red light emitters (and/or light emitters of other wavelengths, such as ultraviolet, near infrared light, etc.).

FIG. 7C is a schematic diagram of an example illumination source 770 including multiple light emitters 772, 774 collectively emitting substantially collimated light towards a focusing optic 116 that focuses the light onto an optical fiber 104, in accordance with various embodiments. The light emitters 772, 774 may be oriented to emit the light generally parallel to the optical axis 706 of the focusing optic 116, achieving the collimation of the overall beam of light, regardless of whether the “microbeams” emitted by the individual emitters 772, 774 are each collimated, as is the case in certain preferred embodiments, or exhibit some degree of beam divergence, as is the case in other embodiments. As a consequence of the focusing properties of the optic 116, light emitted, and crossing the front focal plane of the focusing optic 116 (corresponding to the Fourier transform plane), at greater radial distances from the optical axis 706 will enter the optical fiber 104 at greater input angles. In some embodiments, the light emitters 772, 774 are mounted on a common planar circuit board, with their output facets all arranged along a single plane. As shown, this plane may be positioned to coincide with the front focal plane 776 of the focusing optic 116 (that is, it may be placed at a focal length ƒ preceding the focusing optic 116). As compared with the illumination source 600 depicted in FIG. 6, where the spatial intensity distribution of incoming collimated light is modulated with a light modulator placed in the front focal plane of the focusing optic 116 (which constitutes a Fourier transform plane), illumination source 700 allows controlling the spatial intensity distribution in the front focal plane 776, or Fourier transform plane, directly via the output intensity of the light emitters 772, 774, and thereby controlling the angular intensity distribution of light coupled into the optical fiber 104. Note that, although placing the emitters at the Fourier transform plane may be beneficial (e.g., in that it can simplify the design, manufacturing, and testing process for the illumination source 770), the mapping between the radial distance of the emitter from the optical axis 706 and the input angle to the optical fiber 104 will be, for emitter outputs parallel to the optical axis 706, be at least in principle the same regardless of where along the optical axis 706 the emitters 772, 774 are placed, as will be readily appreciated by those of ordinary skill in the art.

As in the multi-emitter embodiments of FIGS. 7A and 7B, the output intensity of the individual light emitters 772, 774 can be varied by directly changing the optical output power of each emitter (e.g., by adjusting the electrical power supplied to a diode laser, or adjusting the gain of a pumped laser via the power to the pump laser), or by using a controllable amplitude modulator at the laser output to variably reduce the intensity. Further, as described above with respect to illumination sources 700, 750, the light emitters 772, 774 may include groups of emitters that generate light at multiple respective wavelengths (e.g., with a group of emitters 772 emitting green light and a group of emitters 774 emitting blue light), allowing those wavelengths to be combined at the fiber input with minimal or no optics. Within each group, the light emitters 772 or 774 can be independently varied in output intensity, which allows controlling the relative intensities of light at various input angles, and thus the angular intensity distribution at the fiber input, separately for each wavelength.

FIGS. 7D-7F are schematic diagrams of example planar arrangements of light emitters of the illumination source 770 (e.g., in the front focal plane 776), in accordance with various embodiments. In the configuration 780 of FIG. 7D, light emitters 772 (only a couple being labeled) all generating output light at the same wavelength are arranged along concentric circles 782 in the plane. Each circle 782 of emitters maps onto a specific input angle at the input of the optical fiber 104. The emitters 772 along each circle 782 may be controlled together as a group to generate a desired intensity of light at the corresponding input angle to the optical fiber 104, and the relative output intensities between the groups may be controlled to achieve the desired angular intensity distribution at the fiber input. The configuration 784 of FIG. 7E includes light emitters 772, 774, 786 emitting at three different wavelengths (e.g., red, green, and blue) arranged at concentric circles 788, where each circle 788 includes emitters 772, 774, 786 for all three wavelengths. Thus, each group of emitters for a given wavelength includes emitters at different radial distances from the center (where the optical axis 706 is), facilitating control over the angular intensity distribution at that wavelength. FIG. 7F shows a star configuration 790 of light emitters in which, for each of multiple wavelengths, a group of emitters outputting light at that wavelength (e.g., emitters 792, or emitters 794) is arranged along a radius, spamming a range of radial distances from the center (or optical axis 706) that maps onto a range of input angles for controlling the angular intensity distribution at that wavelength.

FIG. 7G is a schematic diagram of an example illumination source 796, otherwise as shown in FIG. 7C, which combines light at multiple wavelengths emitted by groups of light emitters 797, 798 placed at multiple respective planes, in accordance with various embodiments. One group of light emitters 797 may be placed on the optical axis 706 of the focusing optic, e.g., as shown, at a focal length ƒ preceding the focusing optic 116, and emit light in a first direction, parallel to the optical axis 706 directly towards the focusing optic 116. Additional groups of light emitters 798 may be oriented to emit light in one or more second directions, at angles relative to the optical axis 706 (e.g., perpendicularly to the optical axis 706) towards the optical axis 706, and mirrors 799 placed along the optical axis 706, within the beams of light emanating from the groups of light emitters 798, may redirect those beams along the optical axis 706 towards the focusing optic 116. In some embodiments, the groups of light emitters 797, 798 and mirrors 799 are configured such that the mirrors 799 do not occlude the light propagating along the optical axis 706. For example, with the optical axis 706 lying within a horizontal plane, the light emitters 797, 798 may each be arranged linearly along different respective directions relative to a horizontal direction (e.g., as shown in FIG. 7F for emitters 792 and emitters 794, respectively). Alternatively, if the microbeams from emitters 797, 798 spatially overlap, the mirrors 799 may be dichroic mirrors 799 each configured for high reflectivity at the wavelength of the group of emitters 798 with which it is associated, and for high transmissivity of light at the wavelength of the emitters 797 placed on the optical axis 706 and the wavelength of any of the other emitters 798 that precede the mirror 799 on the optical axis 706.

Each group of light emitters 798 may be arranged in a respective plane, and an optical axis associated with the group of light emitters 798 may be defined as the axis normal to the respective plane that meets the optical axis 706 of the focusing optic 116 in the plane of the associated mirror 799. (The axis associated with the group of light emitters 798, together with the segment of the optical axis 706 from the mirror 799 to the focusing optic 106, may also be thought of as a “folded” optical axis.) Within each plane, the emitters 798 may be arranged, e.g., along concentric circles centered at the respective optical axis (as depicted in FIG. 7D), or generally in any way that spans a range of radial distances from the optical axis associated with the group of emitters 798. As shown, each group of emitters 798 may be placed such that the sum of its distance from the associated (e.g., dichroic) mirror 799 and the distance of that mirror 799 from the focusing optic 116 amounts to the focusing length 116 ƒ of the focusing optic, such that the plane in which the emitters 798 are located is effectively, in its optical function, a front focal plane of the focusing optic 116. However, as noted above, a particular optical distance between the light emitters 798 and the focusing optic 116 is not necessary, and the same mapping between radial distance from the optical axis and input angle to the optical fiber 104 is achieved regardless, as long as the emitters 798 emit light in parallel to their respective optical axis.

The illumination source 796 can, in principle, combine an arbitrary number of wavelengths. For example, as shown, it may combine four wavelengths, which may correspond, for example and without limitation, to red, blue, green, and ultraviolet light. Light emitters generating light at different wavelengths may be arranged separately in different respective planes, as shown. It is also possible, however, to combine emitters for a subset of the multiple wavelengths in a single plane, e.g., in the manner shown in FIG. 7E or 7F, For example, light emitters 797 arranged in a plane along the optical axis 706 of the focusing optic 116 may include emitters of red light and emitters of green light, and light emitters 798 arranged in a separate plane, whose output is reflected onto the focusing optic 116 by a mirror 799, may include emitters of blue light and emitters of ultraviolet light. Those of ordinary skill in the art will know how to combine the features described with respect to FIGS. 7C-7G in various ways.

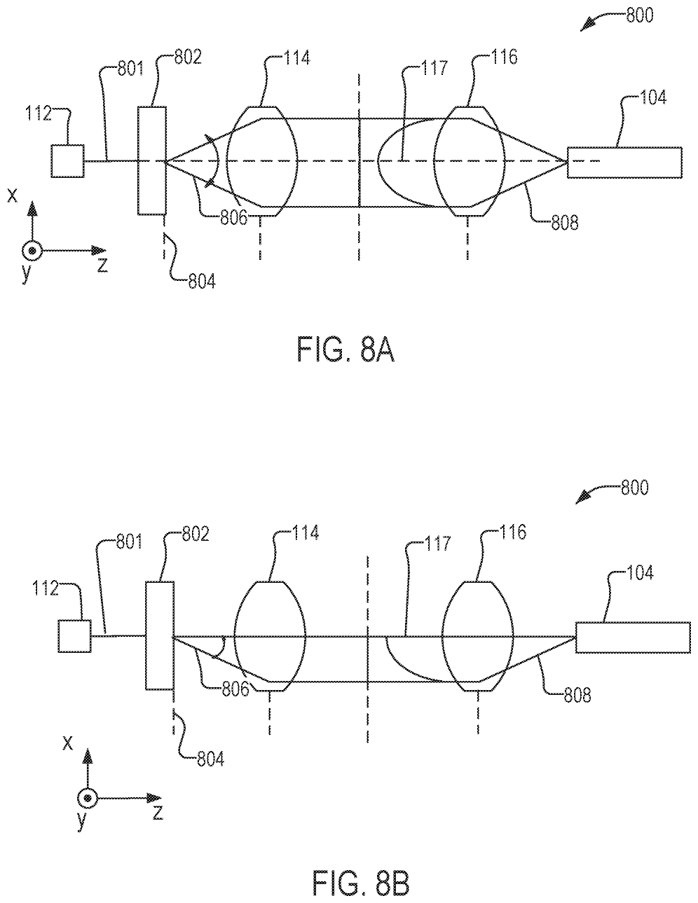

FIGS. 8A and 8B are schematic diagrams of an example illumination source 800 for controlling the angular intensity distribution of light coupled into an optical fiber 104 by scanning light 801 from one or more light emitters 112 across the collimating optic 114 as the intensity is varied, in accordance with various embodiments. As shown, collimated light 801 from the light emitter(s) 112, as it propagates along the optical axis 117, is intercepted by a beam sweeper 802 placed at the front focal plane 804 of the collimating optic 114. The beam sweeper 802 changes the direction of propagation of the light as a function of time, thereby scanning a light beam 806 exiting the beam sweeper 802 (fully or partially) across the surface of the collimating optic 114. The angle relative to the optical axis 117 at which the beam 806 enters the collimating optic 114, herein also the “scanning angle,” will result in the same angle relative to the optical axis 117 of the beam 808 exiting the focusing optic 116 if the focusing lengths of the collimating and focusing optics 114, 116 are the same. In this manner, the light beam 808 coupled into the optical fiber 104 can be scanned across a range of input angles by scanning the light beam 806 entering the collimating optic 114 across that same range of angles.

In some embodiments, the beam sweeper 802 is a “single-axis” beam sweeper that scans the light beam 806 in only one transverse dimension (in one direction perpendicular to the optical axis 117 and direction of propagation of the incoming beam). For example, denoting the direction of light propagation along the optical axis 117 as z, a single-axis beam sweeper may be oriented to scan the beam 806 across the collimating optic 114 in the x-direction; the beam 806 propagates, in this case, in the x-z plane. In some embodiments, the beam sweeper 802 is a “dual-axis” beam sweeper that allows scanning the light beam 806 in two transverse dimensions, that is, across the x-y-plane, by deflecting the incoming light beam into any arbitrary direction. Since the optical fiber 104 itself tends to create an annular output even if light enters the fiber 104 from only one direction, a scan along a line across the surface of the collimating optic 114, intersecting the optical axis 117 may suffice in many cases. Further, the scan may be limited to a line segment between normal incidence onto the collimating optic and a maximum desired input angle. Such a partial scan is depicted in FIG. 8B.

The beam sweeper 802 may be implemented by any of various devices known to those of skill in the art. In some embodiments, one or more acousto-optic modulators (AOMs) are used. AOMs use the acousto-optic effect to diffract light using sound waves generated, for example, by a piezoelectric transducer attached to a plate made of glass or some other material transparent to light. The sound waves create a moving, periodic refractive index modulation in the glass that causes incoming light to scatter and interfere in a manner similar to Bragg diffraction. The diffraction angle depends on the frequency of the sound waves, and the amount of light diffracted at that angle depends on the intensity of the sound. Thus, using an AOM as the beam sweeper 802, the diffraction angle, which corresponds to scanning angle between the beam 806 and the optical axis 117, and the intensity of the beam 806 can be adjusted in a coordinated fashion by simultaneously controlling the sound frequency and intensity, e.g., via the frequency and amplitude of vibrations generated by the piezoelectric transducer. With a single AOM, oriented such that the sound waves propagate in a direction perpendicular to the optical axis 117, diffraction in that direction can be achieved. For example, denoting the direction of light propagation along the optical axis 117 as z and the direction of the propagation of the sound waves as x, the diffracted beam will propagate in a direction in the x-z plane. To achieve diffraction in an arbitrary direction to scan the beam 806 across the x-y plane, two crossed AOMs, one oriented in the x direction and one oriented in the y-direction, may be used.

In an alternative embodiment, one or more mirror galvanometers serve as the beam sweeper 802. A mirror galvanometer includes a mirror that rotates along with a current-carrying coil placed in a magnetic field, deflecting a light beam reflected off the mirror as the mirror rotates. Traditionally used to measure the current in the coil via the deflection of the light, mirror galvanometers are now also commonly employed to Move laser beams (e.g., in laser shows). In the illumination source 800, a mirror galvanometer, or more generally an electrically driven rotating mirror, can be used to deflect incoming light at an electrically controllable angle. As with AOMs, a single rotating mirror allows scanning the beam along one transverse direction, whereas two crossed rotating mirrors achieve full scanning flexibility in both transverse directions. Unlike AOMs, however, rotating mirrors (e.g., in mirror galvanometers) do not themselves modify the intensity of the light.

When using a beam sweeper 802 that changes merely the angle of the light relative to the optical axis 117, the output intensity of the light emitters may be varied (directly, or indirectly via an amplitude modulator at the emitter output) in synchronization with the scanning angle to effect the desired angular intensity distribution of light coupled into the optical fiber 104. For example, the system controller and data processor 124, or a separate controller, may simultaneously control the light emitter(s) 112 (or associated amplitude modulators) and the beam sweeper 802 in accordance with a desired functional dependence (e.g., cosine) between intensity and angle, as may be stored in memory of the system controller and data processor 124. Alternatively, the light emitter(s) 112 may be controlled based on a signal received from the beam sweeper 802 and/or vice versa, or both light emitters 112 and beam sweeper 802 may execute predetermined (e.g., linear or sinusoidal) sweeps of the light intensity and angle, respectively, with trigger signals serving to synchronize the sweeps.

As noted, the beam sweeper 802 allows scanning the beam 806 in one or two dimensions, depending on whether, for instance, only a single AOM or mirror galvanometer, or a pair of crossed AOMs or galvanometers, is used. Since the optical fiber 104 itself tends to create an annular output even if light enters the fiber 104 from only one direction, a scan along a line across the surface of the collimating optic 114, intersecting the optical axis 117, may suffice in many cases. Further, the scan may be limited to a line segment between normal incidence onto the collimating optic and a maximum desired input angle.

While the illumination sources 600, 700, 750 that utilize spatial filtering in the transform plane or multiple light emitters oriented at different angles relative to the optical axis generate a focused input beam to the optical fiber that simultaneously includes light at multiple input angles, the beam sweeper-based illumination source 800 temporally spreads out the input at various angles. An overall uniform perceived illumination of the target can be achieved via such scanning if the scan rate is at least equal to, and coordinated with, the image acquisition rate, such that each image acquired by the sensor aggregates light received over a full scan period or an integer multiple of the scan period (understood to be the period of a fill scan in one direction). In various embodiments, image acquisition rates are between 30 frames per second and 120 frames per second, and scan rates are between 300 Hz and 12 kHz. With scanning illumination, a full-frame readout of the sensor will preferably be used.

A shutter may prevent light from reaching the sensor during read-out, as well as, in cases where multiple successive sweeps are performed between readouts to accumulate enough photons, during turn-around periods of the beam sweeper associated with a change in the scanning direction (if the scan is bi-directional), or during periods in which the beam sweeper is set back to the starting position (if the scan is uni-directional). The system controller and data processor 124 may control the operation of the light emitter(s) 112, beam sweeper 802, sensor 120, and shutter(s) simultaneously and in a coordinated manner.

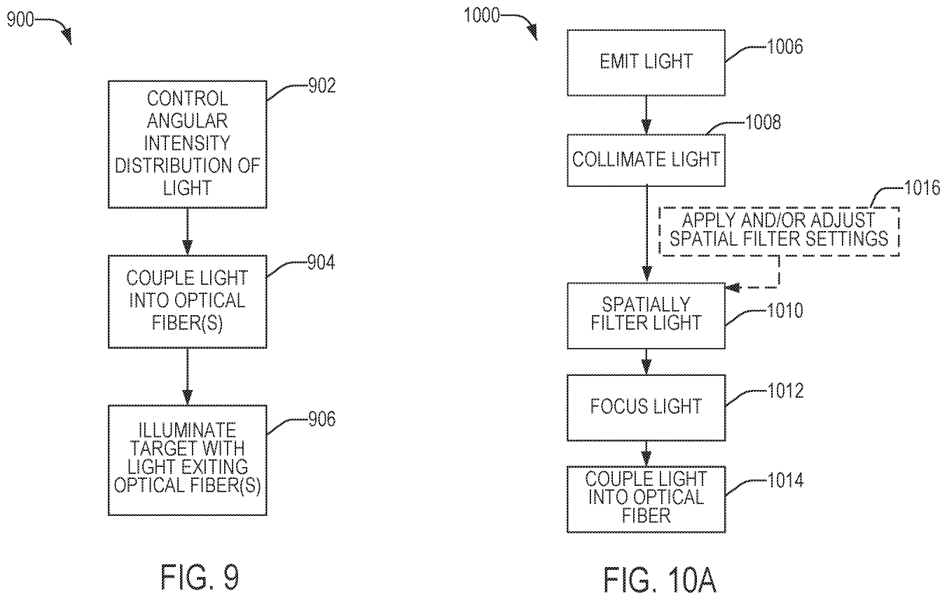

FIG. 9 is a flow chart illustrating a general method 900 of illuminating a target, in accordance with various embodiments. The method 900 employs an illumination source (e.g., 600, 700, 750, 800) to couple light at multiple angles into one or more optical fibers at the input end (in 902) while controlling the angular radiant intensity distribution of the light (in 904), and using the optical fiber(s) to illuminate the target with light exiting the one or more optical fibers at the output end (in 906). The input angular radiant intensity distribution of the light coupled into the fiber may be controlled to cause the output angular radiant intensity distribution of the light exiting the optical fiber to exhibit an increase in output intensity with increasing output angle. The various steps 902, 904, 906, although depicted in sequence to illustrate how the light propagates along its path, are generally performed continuously and in parallel over a period of time.

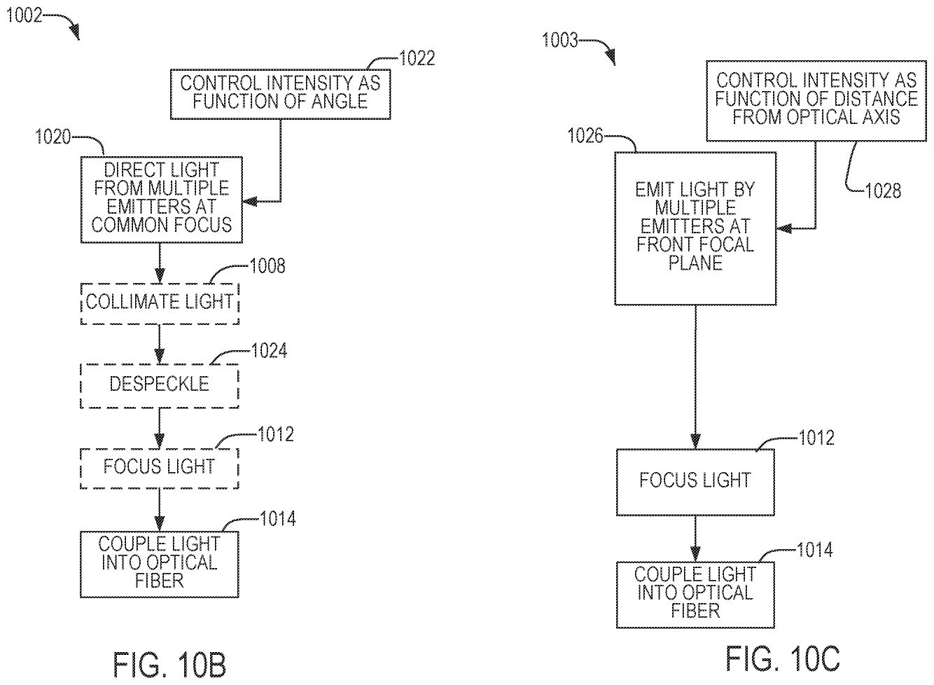

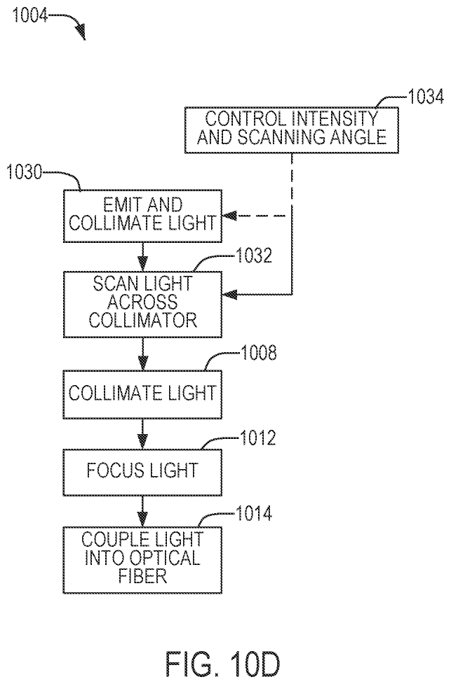

FIGS. 1.0A-10D are flow charts illustrating various methods 1000, 1002, 1003, 1004 for generating a controlled angular radiant intensity distribution at the input to the optical fiber, in accordance with various embodiments. These methods 1000, 1002, 1003, 1004 can be used to achieve perceived uniform illumination. Functionally equivalent steps are given the same reference number across FIGS. 10A-10D.

With reference to FIG. 10A, method 1000, which can be performed with an illumination source 600 as depicted in FIG. 6, involves generating and emitting light by one or more light emitters (in 1006), collimating the light (in 1008), spatially filtering (in 1010) and refocusing (in 1012) the light to achieve a desired intensity distribution as a function of angle, and coupling the focused light into the optical fiber at its input end (in 1014). The spatial filtering (in 1010), which may take place in the Fourier transform plane between collimating and focusing optics, may be performed by a static spatial filter (e.g., a diffractive element or gradient-absorbing filter) or a programmable spatial filter (e.g., an electronically controlled liquid-crystal filter). In the case of a programmable spatial filter, the method 1000 may include a feedback control loop in which the spatial filter is adjusted (in 1016), e.g., via adjustments to the control voltages applied to liquid-crystal elements, based on measurements of the output of the illumination source 600 the output of the optical fiber 104, and/or the images of the illuminated target acquired by the camera's image sensor 120. Such adjustments may be performed pre-deployment of the illumination source 600 as part of a calibration procedure utilizing a calibration target to optimize voltage settings of the spatial filter, and the determined settings may then be applied during subsequent use of the illumination source 600. Alternatively or additionally, adjustments may be made in situ as the illumination source 600 is used in an end application to illuminate, e.g., an anatomical target, to fine-tune the spatial filter for improved illumination uniformity.

With reference to FIG. 10B, in method 1002, which can be performed with an illumination source 700 or 750 as depicted in FIGS. 7A-7B, light from multiple emitters is directed at a common focus (in 1020), The emitters are oriented such that they emit light at multiple different angles relative to the optical axis, and the intensities of the emitters are individually controlled as a function of angle (in 1022) to achieve a desired angular intensity distribution. Intensity control can be achieved by controlling the function of the emitter, or by modulating the light output by the emitters with a separate optical modulator device. The common focus of the multiple emitters is, in some embodiments, placed at the input end of the optical fiber to directly couple the light into the optical fiber (in 1014). In other embodiments, the light output by the emitters is collimated (in 1008), despeckled (in 1024), and refocused (in 1012) before being coupled into the optical fiber (in 1014); in that case, the common focus may be located at the front focal plane of the collimating optic.

With reference to FIG. 10C, in method 1003, which can be performed with an illumination source 770 or 796 as depicted in FIGS. 7C-7G, collimated light is emitted by multiple emitters, e.g., placed at a front focal plane (or multiple front focal planes) of a focusing optic (in 1026), and the emitted light is focused, by the focusing optic, onto the input of the optical fiber (in 1012). The emitters are placed at multiple different radial distances from the optical axis (e.g., the optical axis of the focusing optic, or an optical axis associated with the light emitters if a mirror in the beam path redirects the light), and the intensities of the emitters are individually controlled as a function of radial distance (in 1028) to achieve a desired angular intensity distribution. Intensity control can be achieved by controlling the function of the emitter, or by modulating the light output by the emitters with a separate optical modulator device.

With reference to FIG. 1.013, method 1004, which can be performed with an illumination source 800 as depicted in FIGS. 8A-8B, involves generating a generally collimated beam of light by one or more emitters (in 1030), and scanning the collimated beam across the surface of a (downstream) collimating optic (in 1032). The collimating optic generates a collimated beam of light parallel to the optical axis (in 1008), at a distance from the optical axis that depends on the momentary scanning angle and place where the scanned beam hits the surface of the collimating optic. The collimated light leaving the collimating optic at any distance from the axis is focused (in 1012) at the input end of the optical fiber, where it is coupled into the optical fiber (in 1014). In order to achieve the desired angular intensity distribution at the input to the optical fiber, the scanning angle and intensity of the light scanned across the collimating optic may be controlled simultaneously and in a coordinated manner (1.034) by controlling the scanning angle of the beam sweeper in conjunction with the output intensity of the emitter(s) or an intensity modulation inherently applied by the beam sweeper.

Various approaches to controlling the angular intensity distribution of light coupled into an optical fiber have been described. In various embodiments, the disclosed apparatus (e.g., illumination sources 600, 700, 750, 800) is operated within a fiber-based image capture system (e.g, system 100) to achieve perceived uniform illumination of a target, as reflected in a measured intensity across the captured image that is uniform except for any variations due to varying surface properties of the imaged target. Specifically, the angular intensity distribution at the fiber input may be adjusted to compensate for the radial fall-off in intensity observed in conventional systems that output substantially isotropic light at the output end of the optical fiber. Beneficially, the technical effect of uniform illumination can be achieved at the image sensor, without any need for electronically compensating the sensor output signals. Thus, the benefits of uniform perceived illumination, which include improved accuracy in measuring the fluorescence or absorbance behavior of the target surface, can be realized without the technical problems associated with electronic compensation, such as increased and/or spatially non-uniform electronic noise.

The following numbered examples are illustrative embodiments.

1. A method for illuminating a target, the method comprising: at an input end of one or more optical fibers, coupling light into the one or more optical fibers at multiple input angles relative to an optical axis of the one or more optical fibers at the input end; illuminating the target with light exiting the one or more optical fibers at an output end of the one or more optical fibers; and controlling an input angular radiant intensity distribution of the light at the input end to cause an output angular radiant intensity distribution exhibiting an increase in output intensity with increasing output angle.

2, The method of example 1, wherein the input angular radiant intensity distribution of the light at the input end is controlled to achieve uniform perceived illumination of the target as measured by a camera.

3. The method of example 1 or example 2, wherein the camera is substantially collocated with the output end of the one or more optical fibers.

4. The method of any of examples 1-3, wherein the target comprises an anatomical target, the output end of the one or more optical fibers is placed inside a patient's body, and the light at the input end is coupled into the one or more optical fibers at the input end outside the patient's body.

5. The method of any of examples 1-4, wherein the one or more optical fibers form a fiber bundle.

6. The method of any of examples 1-5, wherein controlling the input angular radiant intensity distribution of the light at the input end comprises increasing a radiant intensity of the light at the input end with increasing input angles.

7. The method of any of examples 1-6, wherein coupling the light into the one or more optical fibers comprises collimating light emitted by one or more light emitters and focusing the collimated light onto the input end of the one or more optical fibers.

8. The method of example 7, wherein controlling the input angular radiant intensity distribution of the light at the input end comprises controlling the collimated light in a transform plane.

9, The method of example 8, wherein controlling the collimated light in the transform plane comprises spatial filtering in the transform plane.

10. The method of example 9, wherein the spatial filtering is performed using one of a diffractive element with a static phase profile or a gradient-absorbing filter with a static absorption profile.

11. The method of example 9, wherein the spatial filtering comprises adjusting controllable elements of a programmable spatial filter.

12. The method of example 11, wherein adjusting the controllable elements comprises adjusting electrical voltages applied to electrically addressable liquid crystal regions.

13. The method of example 11 or example 12, wherein adjusting the controllable elements comprises adjusting the controllable elements based at least in pad: on measurements of at least one of the input angular radiant intensity distribution at the input end of the one or more optical fibers, the output angular radiant intensity distribution at the output end of the one or more optical fibers, or an illumination of the target.

14. The method of any of examples 1-6, wherein coupling the light into the one or more optical fibers comprises directing light emitted by multiple light emitters onto the input end of the one or more optical fibers.

15. The method of example 14, wherein controlling the input angular radiant intensity distribution of the light at the input end of the one or more optical fibers comprises controlling relative output intensities of the multiple light emitters.

16. The method of example 15, wherein the relative output intensities of the multiple light emitters are controlled based at least in part on measurements of at least one of an input angular radiant intensity distribution at the input end of the one or more optical fibers, an output angular radiant intensity distribution at an output end of the one or more optical fibers, or an illumination of the target.

17. The method of any of examples 14-16, wherein the multiple light emitters comprises one or more groups of light emitters emitting light at different respective wavelengths.

18. The method of any of claims 14-17, wherein the multiple light emitters are oriented to emit light towards a focal region at a front focal plane of a collimating optic at multiple angles with respect to an optical axis of the collimating optic, and wherein directing the light onto the input end of the one or more optical fibers comprises focusing the collimated light onto the input end of the one or more optical fibers.

19. The method of example 18, further comprising despeckling the collimated light at a transform plane between the collimating optic and a focusing optic used to focus the collimated light onto the input end of the one or more optical fibers.

20. The method of any of examples 14-17, wherein the multiple light emitters are oriented to emit the light towards the input end of the one or more optical fibers at the multiple input angles.

21. The method of example 20, wherein the multiple light emitters comprise one or more groups of light emitters, each group arranged in a plane comprising the optical axis of the one or more optical fibers at the input end.

22. The method of any of examples 14-17, wherein the multiple light emitters are oriented to emit substantially collimated light, and wherein directing the light onto the input end of the one or more optical fibers comprises focusing the collimated light onto the input end of the one or more optical fibers.

23. The method of example 22, wherein the multiple light emitters are arranged at a front focal plane of a focusing optic focusing the collimated light onto the input end of the one or more optical fibers.

24. The method of example 22 or example 23, wherein the multiple light emitters comprise, arranged in single plane, multiple groups of light emitters emitting light at different respective wavelengths.

25. The method of example 22 or example 23, wherein the multiple light emitters comprise a first group of light emitters emitting substantially collimated light at a first wavelength in a first direction towards a focusing optic that focuses the collimated light onto the input end of the one or more optical fibers, the method further comprising emitting substantially collimated light at a second wavelength different from the first wavelength in a second direction different from the first direction, and redirecting the substantially collimated light at the second wavelength from the second direction to the first direction towards the focusing Optic.

26. The method of any of claims 1-6, wherein coupling the light into the one or more optical fibers comprises scanning light from one or more light emitters across a collimating optic that generates collimated light, and focusing the collimated light onto the input end of the one or more optical fibers.

27. The method of example 26, wherein controlling the input angular radiant intensity distribution of the light at the input end comprises coordinating an intensity of the scanned light with a scanning angle of the scanned light.

28. The method of example 27, wherein the intensity and scanning angle of the scanned light are controlled via a diffraction angle and strength of diffraction in one or more acousto-optic modulators.

29. The method of example 27 or example 28, wherein coordinating an intensity of the scanned light with a scanning angle of the scanned light comprises controlling an output intensity of the one or more light emitters based on a scanning angle of the scanned light.

30. The method of any of examples 26-29, wherein the light is scanned across the collimating optic in one dimension.

31. The method of any of examples 26-29, wherein the light is scanned across the collimating optic in two dimensions.

32. An illumination source comprising: one or more light emitters configured to emit light; a collimating optic positioned to collimate the light emitted by the one or more light emitters; a focusing optic positioned to focus the collimated light into a focal region operably collocated with an input end of one or more optical fibers; and a light modulator positioned at a transform plane between the collimating and focusing optics, the light modulator configured to cause an intensity of light in the focal region to vary as a function of input angle to the one or more optical fibers.

33. The illumination source of example 32 wherein the light modulator comprises a static modulator.

34. The illumination source of example 33, wherein the light modulator comprises a diffractive element or a gradient-absorbing filter.

35. The illumination source of example 32, wherein the light modulator is programmable.

36. The illumination source of example 35 wherein the light modulator comprises multiple individually addressable elements having variable controllable transmissivity.

37. The illumination source of example 36, wherein the light modulator further comprises electronic circuitry for controlling the transmissivity of the individually addressable elements.

38. The illumination source of example 36 or example 37, wherein the elements comprise a liquid crystal material.

39. The illumination source of claim 32, wherein the light modulator has a spatially varying phase profile or transmission profile.

40. The illumination source of example 39, wherein the phase profile or transmission profile varies radially about an optical axis.

41. An illumination source comprising: a plurality of light emitters configured to emit light towards a common region at different angles relative to an optical axis of the illumination source; and a controller configured to vary, based on the angles, relative intensities of the emitted light.

42. The illumination source of example 41 wherein the common region is operatively collocated with an input end of one or more optical fibers.

43. The illumination source of example 41 or example 42, further comprising: a collimating optic positioned to collimate the light emitted by the plurality of light emitters; and a focusing optic positioned to focus the collimated light into a focal region operably collocated with an input end of one or more optical fibers, wherein the common region is placed at a front focal plane of the collimating optic.

44. The illumination source of example 43, further comprising: a speckle reducer placed at a transform plane between the collimating and focusing optics.

45. The illumination source of any of example 41-44, wherein the controller is configured to vary the relative intensities of the emitted light by controlling at least one of output intensities produced by the plurality of light emitters or intensity reductions imparted by amplitude modulators at outputs of the plurality of light emitters.

46. The illumination source of any of examples 41-45, wherein the plurality of light emitters comprise two or more groups of light emitters, a first group of light emitters of the two or more groups of light emitters configured to emit light of a first wavelength and a second group of light emitters of the two or more groups of light emitters configured to emit light of a second wavelength different from the first wavelength.

47. The illumination source of example 46, wherein the first group of light emitters is arranged in a first half plane including the optical axis and the second group of light emitters is arranged in a second half plane including the optical axis, wherein the second half plane is different from the first half plane.

48. An illumination source comprising: a plurality of light emitters configured to emit substantially collimated light; a focusing optic positioned to focus the light emitted by the plurality of light emitters into a focal region operably collocated with an input end of one or more optical fibers; and a controller configured to vary relative intensities of the light emitted by the plurality of light emitters based in part on a radial distance of the light emitters from an optical axis.

49. The illumination source of example 48, wherein the common region is operatively collocated with an input end of one or more optical fibers.

50. The illumination source of example 48 or example 49, wherein the controller is configured to vary the relative intensities of the emitted light by controlling at least one of output intensities produced by the plurality of light emitters or intensity reductions imparted by amplitude modulators at outputs of the plurality of light emitters.

51. The illumination source of any of examples 48-50, wherein the multiple light emitters are arranged in a front focal plane of the focusing optic.

52. The illumination source of any of examples 48-51, wherein the plurality of light emitters comprise two or more groups of light emitters arranged in a common plane, a first group of light emitters of the two or more groups of light emitters configured to emit light of a first wavelength and a second group of light emitters of the two or more groups of light emitters configured to emit light of a second wavelength different from the first wavelength.

53. The illumination source of any of examples 48-51, wherein the plurality of light emitters comprises a first group of light emitters emitting substantially collimated light at a first wavelength in a first direction towards the focusing optic and a second group of light emitters emitting substantially collimated light at a second wavelength different from the first wavelength in a second direction different from the first direction, the illumination source further comprising a mirror redirecting the substantially collimated light at the second wavelength from the second direction to the first direction towards the focusing optic.

54. An illumination source comprising: one or more light emitters configured to emit light; a beam sweeper configured to sweep the light emitted by the one or more light emitters; a collimating optic positioned to collimate the swept light; a focusing optic positioned to focus the collimated light into a focal region operably collocated with an input end of one or more optical fibers; and a controller configured to cause the beam sweeper to sweep the light across the collimating optic and to cause an intensity of the swept light to vary in synchronization with the sweeping.

55. The illumination source of example 54, wherein the controller is configured to control the one or more light emitters to vary the intensity of the emitted light in synchronization with the sweeping.

56. The illumination source of example 54, wherein the controller is configured to control the beam sweeper to vary the intensity of the light exiting the beam sweeper in synchronization with the sweeping.

57. The illumination source of any of examples 54-56, wherein the beam sweeper comprises one or more acousto-optic modulators.

58. The illumination source of any of examples 54-56, wherein the beam sweeper comprises one or more galvanometers.

59. The illumination source of any of examples 54-58, wherein the beam sweeper comprises two crossed devices to sweep the light across the collimating optic in two dimensions.

60. A system comprising: one or more optical fibers having an input end and an output end; a camera positioned to receive light from a target illuminated by light exiting the output end of the one or more optical fibers; and an illumination source configured to couple light into the one or more optical fibers at the input end and to cause the light to vary in intensity based on an input angle to the one or more optical fibers.

61. The system of example 60, wherein the camera is substantially collocated with the output end of the one or more optical fibers.

62. The system of example 60 or example 61, wherein the illumination source is configured to cause an input angular radiant intensity distribution of the light at the input end that exhibits an increase in radiant intensity with increasing input angle.

63. The system of any of examples 60-62, wherein the illumination source comprises: one or more light emitters configured to emit light; a collimating optic positioned to collimate the light emitted by the one or more light emitters; a focusing optic positioned to focus the collimated light into a focal region at the input end of the one or more optical fibers; and a light modulator positioned at a transform plane between the collimating and focusing optics, the light modulator configured to cause the light to vary in intensity based on the input angle to the one or inure optical fibers.

64. The system of example 63, wherein the light modulator comprises one of a diffractive element, a gradient-absorbing filter, or a programmable modulator with multiple individually addressable elements having variable controllable transmissivity.

65. The system of any of examples 60-62, wherein the illumination source comprises: a plurality of light emitters configured to emit light towards the input end of the one or more optical fibers at different input angles; and a controller configured to vary relative intensities of the emitted light based on the different input angles.

66. The system of any of examples 60-62, wherein the illumination source comprises: a plurality of light emitters configured to emit light towards a common region at different angles relative to an optical axis of the illumination source, the optical axis of the illumination source coinciding with an optical axis of the one or more optical fibers at the input end; a collimating optic positioned to collimate the light emitted by the plurality of light emitters; a focusing optic positioned to focus the collimated light into a focal region at the input end of the one or more optical fibers; a speckle reducer placed at a transform plane between the collimating and focusing optics; and a controller configured to vary relative intensities of the emitted light based on the different angles relative to the optical axis.

67. The system of any of examples 60-62, wherein the illumination source comprises: a plurality of light emitters configured to emit substantially collimated light; a focusing optic positioned to focus the light emitted by the plurality of light emitters into a focal region operably collocated with an input end of one or more optical fibers; and a controller configured to vary relative intensities of the light emitted by the plurality of light emitters based in part on a radial distance of the light emitters from an optical axis.

68. The system of any of examples 60-62, wherein the illumination source comprises: one or more light emitters configured to emit light; a beam sweeper configured to sweep the light emitted by the one or more light emitters; a collimating optic positioned to collimate the swept light; a focusing optic positioned to focus the collimated light into a focal region operably collocated with an input end of the one or more optical fibers; and a controller configured to cause the beam sweeper to sweep the light across the collimating optic and to cause an intensity of the swept light to vary in synchronization with the sweeping.

69. The system of example 68, wherein the controller is configured to control the one or more light emitters to vary the intensity of the emitted light in synchronization with the sweeping.

70. The system of example 68, wherein the beam sweeper comprises one or more acousto-optic modulators and the controller is configured to control the beam sweeper to vary the intensity of the light exiting the beam sweeper in synchronization with the sweeping.

While the disclosed subject matter has been described and explained herein with respect to various example embodiments, these examples are intended as illustrative only and not as limiting, Various modifications, additional combinations of features, and further applications of the described embodiments that do not depart from the scope of the subject matter may occur to those of ordinary skill in the art. Accordingly, the scope of the inventive subject matter is to be determined by the scope of the following claims and all additional claims supported by the present disclosure, and all equivalents of such claims.

1. A method for illuminating a target, the method comprising:
at an input end of one or more optical fibers, coupling light emitted by multiple light emitters into the one or more optical fibers at multiple input angles relative to an optical axis of the one or more optical fibers at the input end;
illuminating the target with light exiting the one or more optical fibers at an output end of the one or more optical fibers; and
controlling an input angular radiant intensity distribution of the light at the input end, by controlling relative output intensities of the multiple light emitters, to cause an output angular radiant intensity distribution exhibiting an increase in output intensity with increasing output angle.

2. The method of claim 1, wherein the input angular radiant intensity distribution of the light at the input end is controlled to achieve uniform perceived illumination of the target as measured by a camera.

3. The method of claim 2, wherein the camera is substantially collocated with the output end of the one or more optical fibers.

4. The method of claim 1, wherein the target comprises an anatomical target, the output end of the one or more optical fibers is placed inside a patient's body, and the light at the input end is coupled into the one or more optical fibers at the input end outside the patient's body.

5. The method of claim 1, wherein the one or more optical fibers form a fiber bundle.

6. The method of claim 1, wherein controlling the input angular radiant intensity distribution of the light at the input end comprises increasing a radiant intensity of the light at the input end with increasing input angles.

7-15. (canceled)

16. The method of claim 1, wherein the relative output intensities of the multiple light emitters are controlled based at least in part on measurements of at least one of an input angular radiant intensity distribution at the input end of the one or more optical fibers, an output angular radiant intensity distribution at an output end of the one or more optical fibers, or an illumination of the target.

17. The method of claim 1, wherein the multiple light emitters comprise one or more groups of light emitters emitting light at different respective wavelengths.

18. The method of claim 1, wherein the multiple light emitters are oriented to emit light towards a focal region at a front focal plane of a collimating optic at multiple angles with respect to an optical axis of the collimating optic, wherein the collimating optic generates collimated light from the light at the multiple angles with respect to the optical axis of the collimating optic, and wherein coupling the light emitted by the multiple light emitters into the one or more optical fibers at the multiple input angles relative to an optical axis of the one or more optical fibers at the input end comprises focusing the collimated light onto the input end of the one or more optical fibers.

19. The method of claim 18, further comprising despeckling the collimated light at a transform plane between the collimating optic and a focusing optic used to focus the collimated light onto the input end of the one or more optical fibers.

20. The method of claim 1, wherein the multiple light emitters are oriented to emit the light towards the input end of the one or more optical fibers at the multiple input angles.

21. (canceled)

22. The method of claim 1, wherein the multiple light emitters are oriented to emit substantially collimated light, and wherein coupling the light emitted by the multiple light emitters into the one or more optical fibers at the multiple input angles comprises focusing the collimated light onto the input end of the one or more optical fibers.

23-40. (canceled)

41. An illumination source comprising:
a plurality of light emitters configured to emit light towards a common region at different angles relative to an optical axis of the illumination source; and
a controller configured to vary, based on the angles, relative intensities of the emitted light.

42. The illumination source of claim 41, wherein the common region is operatively collocated with an input end of one or more optical fibers.

43. The illumination source of claim 41, further comprising:
a collimating optic positioned to collimate the light emitted by the plurality of light emitters; and
a focusing optic positioned to focus the collimated light into a focal region operably collocated with an input end of one or more optical fibers,
wherein the common region is placed at a front focal plane of the collimating optic.

44. The illumination source of claim 43, further comprising:
a speckle reducer placed at a transform plane between the collimating and focusing optics.

45. The illumination source of claim 41, wherein the controller is configured to vary the relative intensities of the emitted light by controlling at least one of output intensities produced by the plurality of light emitters or intensity reductions imparted by amplitude modulators at outputs of the plurality of light emitters.

46. The illumination source of claim 41, wherein the plurality of light emitters comprise two or more groups of light emitters, a first group of light emitters of the two or more groups of light emitters configured to emit light of a first wavelength and a second group of light emitters of the two or more groups of light emitters configured to emit light of a second wavelength different from the first wavelength.

47. The illumination source of claim 46, wherein the first group of light emitters is arranged in a first half plane including the optical axis and the second group of light emitters is arranged in a second half plane including the optical axis, wherein the second half plane is different from the first half plane.

48. An illumination source comprising:
a plurality of light emitters configured to emit substantially collimated light;
a focusing optic positioned to focus the light emitted by the plurality of light emitters into a focal region operably collocated with an input end of one or more optical fibers; and
a controller configured to vary relative intensities of the light emitted by the plurality of light emitters based in part on a radial distance of the light emitters from an optical axis.

49. The illumination source of claim 48, wherein the common region is operatively collocated with an input end of one or more optical fibers.

50. The illumination source of claim 48, wherein the controller is configured to vary the relative intensities of the emitted light by controlling at least one of output intensities produced by the plurality of light emitters or intensity reductions imparted by amplitude modulators at outputs of the plurality of light emitters.

51. The illumination source of claim 48, wherein the multiple light emitters are arranged in a front focal plane of the focusing optic.

52. The illumination source of claim 48, wherein the plurality of light emitters comprise two or more groups of light emitters arranged in a common plane, a first group of light emitters of the two or more groups of light emitters configured to emit light of a first wavelength and a second group of light emitters of the two or more groups of light emitters configured to emit light of a second wavelength different from the first wavelength.

53. The illumination source of claim 48, wherein the plurality of light emitters comprises a first group of light emitters emitting substantially collimated light at a first wavelength in a first direction towards the focusing optic and a second group of light emitters emitting substantially collimated light at a second wavelength different from the first wavelength in a second direction different from the first direction, the illumination source further comprising a mirror redirecting the substantially collimated light at the second wavelength from the second direction to the first direction towards the focusing optic.

54-70. (canceled)
